(19) United States
(12) Patent Application Publication
(10) Pub. No.: US 2008/0081997 A1
(43) Pub. Date: Apr. 3, 2008

(54) APPARATUS AND METHOD FOR DIAGNOSING ISCHEMIC HEART DISEASE

(76) Inventor: Ri-ichiro Kakihara, Toyohashi-shi (JP)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/865,299

(22) Filed: Oct. 1, 2007

(30) Foreign Application Priority Data
Oct. 2, 2006 (JP) 2006-271002

(51) Int. Cl. A61B 8/14; G06F 19/00

(52) U.S. Cl. 600/450; 702/19

(57) ABSTRACT

A region of interest is set at a thin layer on the inside of the left ventricular wall for ecocardiographical apical long-axis tomographic images obtained while at rest, and the strain rate of the set region of interest is calculated. The value of a discriminant function is determined on the basis of a plurality of strain rates of an intermediate portion of the systolic phase. Ischemic heart disease is then diagnosed according to the value of the discriminant function.

### FIELD OF THE INVENTION

The present invention relates to an apparatus and a method that diagnose ischemic heart disease by echocardiography.

### BACKGROUND OF THE INVENTION

Conclusive diagnosis of ischemic heart disease is given by coronary angiography. The process of coronary angiography consists of insertion of a narrow catheter into a coronary artery from an arm or the groin, injection of a contrast agent for X-ray imagining, and examination of X-rays from multiple directions. The acquired X-ray images are observed macroscopically, and the percentage of stenosis at each portion of the coronary arteries is examined for the assessment of future treatment. This process is wholly based on the operator's experience. Future treatment is then determined, including whether surgical treatment should be performed on the patient.

As stated above, coronary angiography involves the insertion of a foreign body into a blood vessel, and is often performed even on patients with high degrees of stenosis. Therefore, although being rare cases, serious hemorrhaging or large hematomas may be caused. Also, there is another disadvantage of high health care costs for this procedure.

Considering the given demerits of coronary angiography, it is important to conduct accurate screening in order to assess whether coronary angiography really needs to be performed on each patient. Methods used for such screening include the use of electrocardiogram or the use of ultrasonic echocardiography. In diagnostic methods using an electrocardiography, stress is applied to the patient's heart by physical exercise, and the heart under stress is examined using an electrocardiogram. However, the diagnosis rate when an electrocardiogram is employed is only about 65%, and 10% of these cases, when further assessed by coronary angiography, prove to be cases which require surgical operations.

As for echocardiography tests, one type of method consists of comparing patient's echo images before and after exercise. The process of the other type of method consists of first increasing blood pressure and heart rate by administrating a certain amount of a drug, the dose of which is gradually increased at fixed intervals, and the echo images are monitored throughout the process, while the patients themselves are at rest. In either type of methods, as described, stress is applied to the heart through exercise or drugs, and abnormalities of wall motion of the left ventricle can normally be observed macroscopically. Based on the on-site observation and the past experience of the physician, diagnosis is then made. At present, the method using drugs yields a diagnosis rate of about 85%. However, this method, which strictly relies on images acquired by echocardiography, requires a considerable amount of experience on the part of each physician, in that it is essential yet technically difficult to keep applying adequate stress to heart and obtain images of constant and stable diagnostic sensitivity. Thus, several points being considered, it is clear that there is a potential demand for a method which can diagnose ischemic heart disease objectively and accurately, without either relying on physician experience or placing a physical burden on patients.

### SUMMARY OF THE INVENTION

The present invention relates to a diagnostic apparatus and a diagnostic method that enable ischemic heart disease to be diagnosed without being influenced by technical experience using echocardiography while the patient is at rest.

According to a first aspect of the present invention, a diagnostic apparatus is provided with: a unit for displaying resting apical long-axis ecocardiographical images obtained by an echocardiography imaging unit, a region of interest setting unit for setting the region of interest at a thin layer on the inside of the left ventricular wall in the long-axis echocardiographical images, a strain rate calculation unit for calculating the strain rate of the region of interest, and a discrimination value calculation unit for calculating the value of a predetermined discriminant function based on a plurality of strain rates of an intermediate portion of the systolic phase, wherein ischemic heart disease is diagnosed according to the calculated discrimination value.

According to a second aspect of the present invention, a diagnostic method comprises: setting a region of interest at a thin layer on the inside of the left ventricular wall for echocardiographical apical long-axis tomographic images obtained while at rest, calculating the strain rate of the set region of interest, calculating a predetermined discriminant function on the basis of a plurality of strain rates of an intermediate portion of the systolic phase, and diagnosing ischemic heart disease according to that discrimination value.

According to a third aspect of the present invention, a storage medium has stored thereon a computer program executable to perform the steps of: executing with a computer a step for setting a region of interest at a thin layer on the inside of the left ventricular wall for echocardiographical apical long-axis tomographic images obtained while at rest, a step for calculating the strain rate of the set region of interest, and a step for calculating the value of a predetermined discriminant function based on a plurality of strain rates of an intermediate portion of the systolic phase.

The width of the thin layer on the inside of the left ventricular wall is preferably ⅓ or less of the entire left ventricular wall layer, and the intermediate portion of the systolic phase is preferably within the range of 100 to 200 ms.

Moreover, a unit is preferably provided that calculates an average value from the plurality of strain rates at an intermediate portion of the systolic phase, and ischemic heart disease is preferably diagnosed based on that average value.

Moreover, the plurality of strain rates of an intermediate portion of the systolic phase can consist of the strain rate at the start of the intermediate portion of the systolic phase, the strain rate at the end of the intermediate portion, and the minimum value of the strain rate of the intermediate portion.

Moreover, ischemic heart disease is even more preferably diagnosed based on the aforementioned average value as well as the strain rate at the start of the intermediate portion of the systolic phase, the strain rate at the end of the intermediate portion, and the minimum value of the strain rate of the intermediate portion.

According to the present invention, ischemic heart disease can be diagnosed at a probability roughly equal to that of stress echocardiography without placing a burden on the patient based on echocardiographical images obtained while the patient is at rest.

### BRIEF DESCRIPTION OF THE DRAWINGS

FIG. 1 shows examples of a strain rate curve and strain curve.

FIG. 2 is a drawing showing an example of an embodiment of a diagnostic apparatus according to the present invention.

FIG. 3 is a drawing showing the operational flow of an embodiment of a diagnostic apparatus of the present invention.

FIG. 4 is a drawing showing an example of an echocardiographical image displayed on a display screen of an embodiment of the present invention.

FIG. 5 consists of drawings showing a strain rate curve and corresponding strain curve for explaining strain rate values used in an embodiment of the present invention.

FIG. 6 is a drawing showing the number of cases used in an example of the present invention.

FIG. 7 consists of drawings showing a comparison of 100 ms SR values between a 75% or more stenosis group and a normal group.

FIG. 8 consists of drawings showing a comparison of 200 ms SR values between a 75% or more stenosis group and a normal group.

FIG. 9 consists of drawings showing a comparison of minimum SR values from 100 to 200 ms between a 75% or more stenosis group and a normal group.

FIG. 10 consists of drawings showing a comparison of average SR values from 100 to 200 ms between a 75% or more stenosis group and a normal group.

FIG. 11 consists of drawings showing a comparison of 100 ms SR values between a 90% or more stenosis group and a normal group.

FIG. 12 consists of drawings showing a comparison of 200 ms SR values between a 90% or more stenosis group and a normal group.

FIG. 13 consists of drawings showing a comparison of minimum SR values from 100 to 200 ms between a 90% or more stenosis group and a normal group.

FIG. 14 consists of drawings showing a comparison of average SR values from 100 to 200 ms between a 90% or more stenosis group and a normal group.

FIG. 15 is a table showing the cutoff values, sensitivity and specificity of 100 ms SR values, 200 ms SR values and average and minimum SR values from 100 to 200 ms in the case of stenosis of 75% or more for all three arteries.

FIG. 16 consists of tables showing the cutoff values, sensitivity and specificity of 100 ms SR values, 200 ms SR values and minimum and average SR values from 100 to 200 ms in the case of stenosis of 75% or more for the left anterior discending coronary artery (LAD), the left circumflex coronary artery (LCX) and the right coronary artery (RCA).

FIG. 17 is a table showing the cutoff values, sensitivity and specificity of 100 ms SR values, 200 ms SR values and minimum and average SR values from 100 to 200 ms in the case of stenosis of 90% or more for all three arteries.

FIG. 18 consists of tables showing the cutoff values, sensitivity and specificity of 100 ms SR values, 200 ms SR values and minimum and average SR values from 100 to 200 ms in the case of stenosis of 90% or more for LAD, LCX and RCA.

### DETAILED DESCRIPTION OF THE PREFERRED EMBODIMENTS

In the following paragraphs, an outline of this invention is first presented, followed by explanation of the invention in reference to the drawings.

Cardiac muscle changes such that it shortens in the direction of the long axis and thickens in the direction of the short axis during the systolic phase. Myocardial tissue strain (%), which represents the rate of change of length in the heart wall in a localized region of cardiac muscle, and strain rate (1/s), which is a time differential of strain and represents change of speed of shortening of length of regional cardiac muscle, are known to be better indicators for evaluating local myocardial function than strain. In particular, two-dimensional (2D) strain and strain rate differ from those of one-dimensional tissue velocity image (TVI) which is based on doppler image in that they are not dependent on the angle of ultrasonic beam, are less susceptible to the effects of noise, and are unaffected by surrounding functions, thus enabling them to be superior indicators.

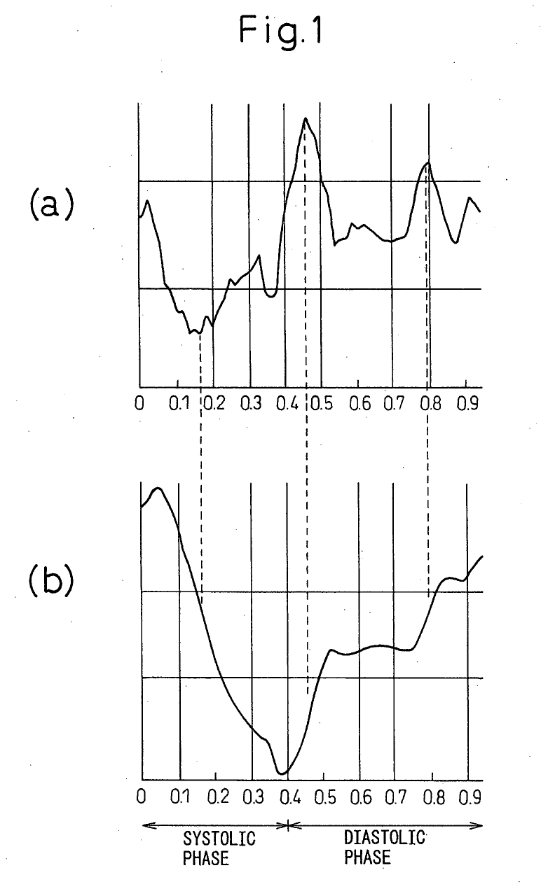

FIG. 1(a) shows a 2D strain rate (to be simply referred to as strain rate) curve in the direction of the long axis for one cardiac cycle of normal heart muscle, while FIG. 1(b) shows a strain curve corresponding to the strain rate curve. Since strain rate is a derivative of strain, a peak value is obtained at the point where the strain slope reaches a maximum. The strain rate reaches a minimum value at nearly the intermediate portion of the systolic period.

Although it is known that strain rate closely reflects the function of a local region of heart muscle, there are cases in which noise gets more emphasized than strain itself, which results in an increased numbers of errors. For this reason, strain rate is now considered as less accurate an indicator than strain itself, and so there has not been many attempts to use strain rate in diagnosing ischemic heart disease such as angina pectoris, in particular.

The inventor has proven that coronary arteries of the heart go into the heart muscle from the epicardium, which is on the outer side of the heart, to the endocardium, which is inside the heart, and therefore, the ends of blood vessels concentrate beneath the endocardium. This means, as the inventor has realized, that decrease of blood flow caused by vasoconstriction will first become most apparent around the endocardium, since that is where ends of blood vessels most concentrate. This clinical phenomenon has not so far been proven echocardiographically because of technical limits of echocardiography. In TVI, sample area in the left ventricular wall is fixed and cannot follow heart muscles in motion, but in 2D strain rate, sample point in ROI is able to follow heart muscle in any direction automatically. Thus the difference of strain rate between the epicardium side and the endocardium side can be observed more accurately. In addition, he has also found that the difference in strain rate of normal regions and stenosis regions are most likely to be seen at the intermediate part of the systolic phase. These significant discovery of facts have lead the inventor to complete the invented system. This invention makes it possible to diagnose ischemic heart disease using the strain rates of patients at rest, by (1) calculating the strain rates concerning a thin region of the endocardium in cross-sectional images taken in the direction of the long axis, and (2) among the calculated strain rates, checking the strain rates of an intermediate part of the systolic phase. Thus, ischemic heart disease can be diagnosed at nearly the same diagnosis rate as the existing methods but without obtaining echo images by placing stress on patients.

The following provides an explanation of an embodiment of the present invention with reference to the drawings.

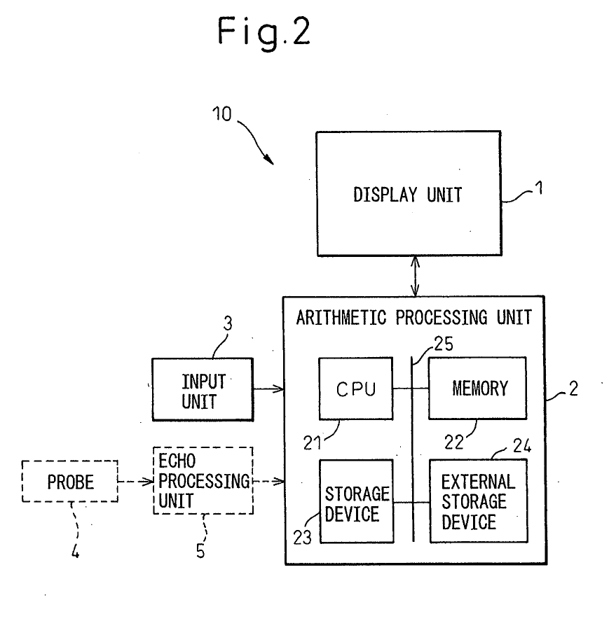

FIG. 2 shows a block drawing of an embodiment of the present invention in the form of an ischemic heart disease diagnostic apparatus using echocardiographical images. Echocardiographical images are generated from reflex signals from the body in the form of ultrasonic echo signals by contacting an ultrasound probe directly with a patient and transmitting ultrasonic waves into the body. The diagnostic apparatus 10 has a display unit 1, which displays echocardiographical images together with graphs or data related thereto, an arithmetic processing unit 2, which carries out image processing and various arithmetic processing, and an input unit 3 for performing user operations or inputting instructions.

Display unit 1 is composed of a graphic display device such as a CRT or liquid crystal display device capable of displaying color images. In addition, display unit 1 composes a user interface in the same manner as input unit 3, enabling interactive operation of diagnostic apparatus 10 by a user. For example, after measurement parameters and measurement sites have been set on the display screen by a user for a tomographic image displayed on display unit 1, the arithmetic processing unit obtains measurement results by calculating tomographic image data based on the set measurement parameters and measurement sites, and those measurement results can be displayed on display unit 1.

Arithmetic processing unit 2 has a CPU 21, and a memory 22 such as a main memory or graphic memory, a storage device 23 such as a hard disc drive, and an external storage device 24 capable of using a removable storage medium such as a DVD or CD, are connected to CPU 21 by a bus 25.

A program executed by CPU 21 is stored in storage device 23, and various data used when the program is executed by CPU 21 are also stored therein. Moreover, ultrasound image data displayed on display unit 1 is also stored. CPU 21 executes a predetermined data processing by loading the program from storage device 23 into main memory. This data processing includes image processing, and processed images are displayed on display unit 1.

Input unit 3 is composed of, for example, a keyboard or mouse, and user operations or instructions can be input to arithmetic processing unit 2.

Echocardiographical images used in the apparatus 10 may also be echocardiographical images input by means of removable external storage device 24. In addition, ultrasonic images may also be input from an ultrasound diagnostic apparatus (not shown) by wired or wireless means. Moreover, diagnostic apparatus 10 may also be made to operate as one of the functions of an ultrasound diagnostic apparatus having an ultrasound probe. In this case, diagnostic apparatus 10 may be provided with an ultrasound probe 4 and an echo processing unit 5 for processing ultrasound signals obtained from ultrasound probe 4 and converting to image data.

Ultrasound probe 4 has an array of a plurality of ultrasound transducers not shown. Ultrasound probe 4 is used by having a user contact the probe with a patient for which images are to be acquired. Signals obtained from ultrasound probe 4 are input to echo processing unit 5. Echo processing unit 5 processes the echo signals to form image data. Arithmetic processing unit 2 then processes the image data to generate images that are displayed on the display unit.

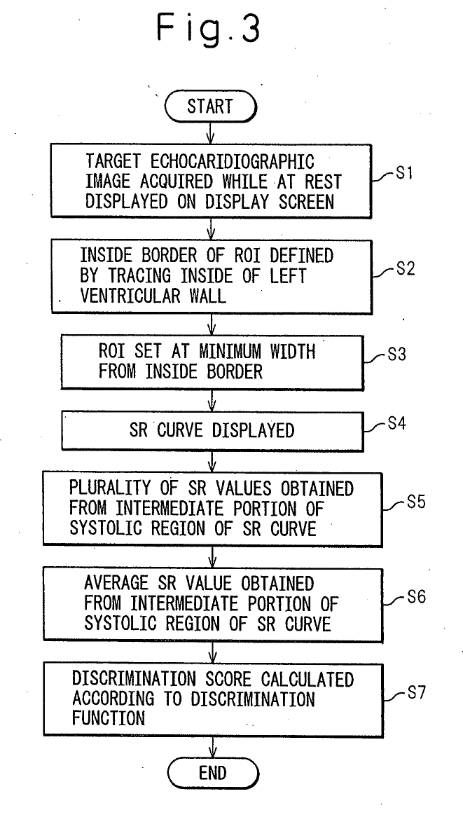

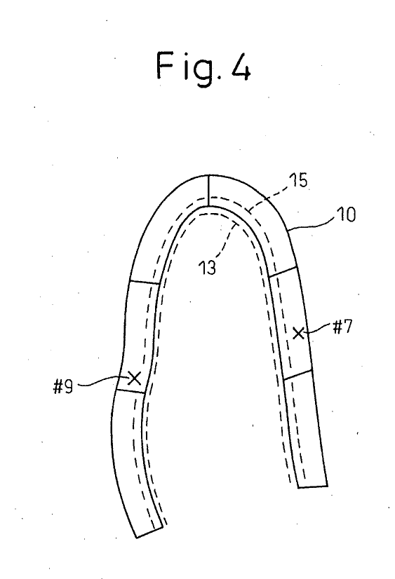

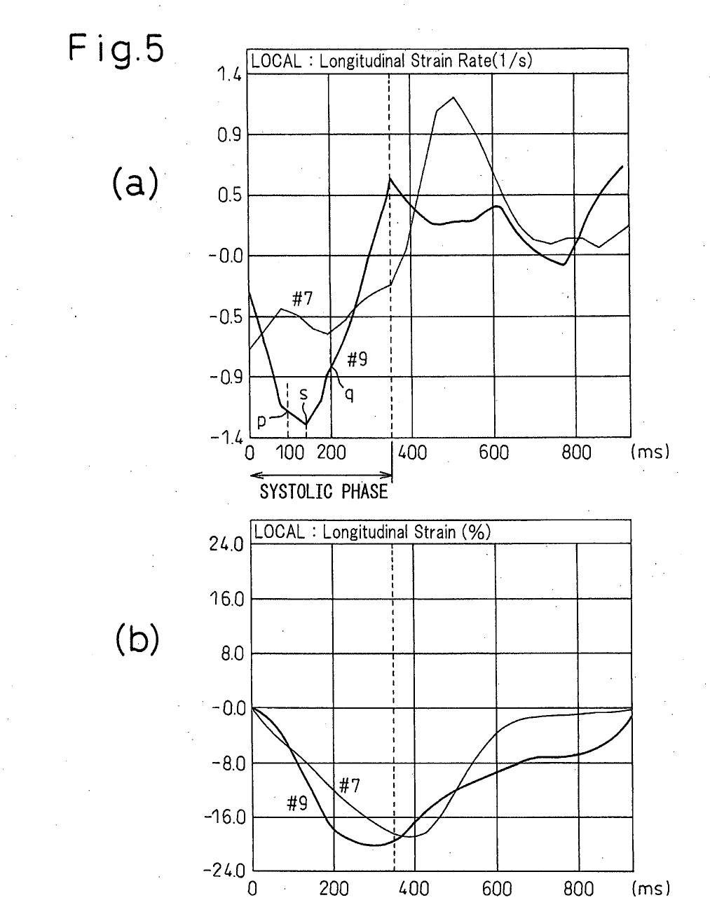

The following provides an explanation of the operation of the diagnostic apparatus according to the present embodiment with reference to FIGS. 3 to 5. FIG. 3 is a flow chart for explaining the operation of the diagnostic apparatus according to the present embodiment, FIG. 4 is a drawing for explaining the operation of setting a region of interest on an echocardiographical image, and FIGS. 5(a) and 5(b) are drawings showing the locations where strain rate values are acquired along with a reference strain curve.

As shown in the flow chart of FIG. 3, in Step S1, an echocardiographical image such as an image of the four chambers of the apical portion of the heart to be analyzed is displayed on the display screen. The echocardiogram to be analyzed is a long-axis cross-sectional image depicting a cross-section in the direction of the long axis viewing from the left ventricular apex. In the examples to be subsequently described in detail, a two-chamber image of the left ventricular, the center of the anterior wall is used to analyze the left anterior descending coronary artery (LAD), and the center of the inferior wall is used to analyze the right coronary artery (RCA), and a long-axis image of the left ventricle the center of the posterior wall is used to analyze the left circumflex coronary artery (LCX).

FIG. 4 shows a schematic cross-sectional view of the left ventricular wall for explaining the tomographic image displayed on the display screen in Step S1. The reason for displaying the cross-section of left ventricular wall 10 in six segments is because the strain rate curve and the like is displayed as six curves corresponding to these segments.

Next, in Step S2, the inside border of a region of interest (ROI) is designated by tracing the inner surface of the endocardium on the image in order to determine the region of interest serving as the region where strain rate is to be measured. In the example shown in FIG. 4, the endocardium of left ventricular wall 10 is traced to designate inside border 13 of the region of interest. Although tracing is performed manually in this example, a program may be incorporated for tracing, the endocardium automatically.

Once the inside border of the region of interest has been demarcated, in Step S3, the outside border of the region of interest is determined so that the region of interest has a thin thickness equal to roughly ⅓ or less the wall thickness of the entire left ventricular wall layer. In this example, the outside border is manually set to the minimum width able to be set for a region of interest. Furthermore, although there may be cases in which the set region of interest is unable to be processed by the arithmetic processing unit or arithmetic processing program in the case of setting manually, in such cases, tracing of the endocardium is redone. This tracing of the endocardium can also be automated by a program. In the example of FIG. 4, the region of interest is the region between inside border 13 and outside border 15.

In Step S4, a strain rate curve for the set region of interest is displayed on the display screen. Normally six strain rate curves are displayed corresponding to the six segments. Obviously only the required number of strain rate curves can also be displayed. FIG. 5(a) shows an example of strain rate curves obtained from the indicated region of interest. However, there is stenosis at site #7 in FIG. 4 while site #9 is normal. It can be determined from FIG. 5(a) that there is a considerable difference in the strain rates between normal site #9 and stenosed site #7 within a range that contains the intermediate portion of the systolic phase and particularly the minimum value. As a result, it can be said that is possible to assess whether or not there is stenosis by checking the strain rate at an intermediate portion of the systolic phase. For reference purposes, FIG. 5(b) shows strain curves at the same sites. The difference between normal site #9 and stenosed site #7 is not as obvious when using strain curves.

Furthermore, the minimum values of normal site #9 and stenosed site #7 are quite different in FIG. 5(a). However, as will be explained later, since strain rate curves have various forms, it is not possible to make a diagnosis based on the minimum value alone.

In Step S5, a plurality of strain rates are obtained from an intermediate portion of the strain rate curves on the display screen. As shown in FIG. 5(a), the strain rate at 100 ms (100 ms SR), the strain rate at 200 ms (200 ms SR) and the minimum value (minimum SR value) of strain rate between 100 and 200 ms are acquired. More specifically, these values are calculated and displayed by designating the SR value at 100 ms, SR value at 200 ms and minimum SR value between 100 and 200 ms with a cursor on the screen on which the strain rate curves are displayed. Furthermore, although the minimum value between 100 and 200 ms is nearly always at an intermediate location between 100 and 200 ms, there are cases in which the minimum value may be obtained at 100 or 200 ms. In such cases, the value at either 100 ms or 200 ms is used as the minimum value.

Next, in Step S6, the average value of strain rate (average SR value) between 100 and 200 ms is determined. Arithmetic processing for determining the average value for a designated interval can be easily executed by providing in advance a program capable of determining an average value of a desired interval. In addition, if the arithmetic processing for determining an average value of a designated interval is made to be executed automatically, the average value between 100 and 200 ms can be determined automatically once 100 ms and 200 ms have been designated in Step S5.

In Step S7, a discrimination score is obtained for diagnosis according to a preprogrammed four-factor discriminant function using the average SR value, 100 ms SR, 200 ms SR and minimum SR value acquired in Step S6. Although specific examples of a discriminant function are explained in the subsequent examples, the discriminant function is composed so that a positive (+) calculated discrimination score indicates stenosis, while a negative (−) discrimination score indicates normal. Since all data between 100 and 200 ms is used, the average value ensures a satisfactory detection probability.

Although specific discriminant functions and resulting detection probabilities will be described later, the use of a four-factor discriminant function using average SR value, 100 ms SR, 200 ms SR and minimum SR value yields a detection probability of more than 86% for stenosis of 75% or more and a detection probability of more than 93% for stenosis of 90% or more.

Furthermore, ischemic heart disease can also be assessed using 100 ms SR, 200 ms SR and minimum SR value without using an average SR value. In this case, after acquiring the 100 ms SR, 200 ms SR and minimum SR values in Step S5, processing skips Step S6 and proceeds to Step S7. In Step S7, a discrimination score is obtained and a diagnosis is made using a three-factor discriminant function using the 100 ms SR, 200 ms SR and minimum SR values.

Since all strain rate data within the range of 100 to 200 ms is used to calculate the average value, this calculation can be bothersome in the case a program for calculating the average value is not provided. Thus, although inferior to a discriminant function using an average value, a three-factor discriminant function is significant for use as a simple diagnostic technique.

### EXAMPLES

The present invention was carried out according to the following examples using actual specific cases including normal and stenosed cases.

The GE Vivid 7 Dimension Version 4.1.0 was used for the ultrasound system, and images were recorded with the patients at rest. Data was analyzed using EchoPAC PC Dimension Version 4.1.0 off-line.

The images used for analysis were comprised of three cross-sections consisting of a four-chamber view (Ap4ch view), long-axis view (ApLax view) and two-chamber view (Ap2ch view) from an apical approach. The center of the left ventricular anterior wall of the two-chamber view was designated as the left anterior descending coronary artery (LAD) region, the center of the left ventricular inferior wall of the two-chamber view was designated as the right coronary artery (RCA) region, and the center of the left ventricular posterior wall of the long-axis view was designated as the left circumflex coronary artery (LCX) region.

After designating LAD for Group A, RCA for Group B and LCX for Group C, a comparative study was conducted between a normal coronary artery group (An, Bn, Cn) and a 75% or more stenosis group (As, Bs, Cs). Moreover, a comparison study was conducted with normal cases for cases having stenosis of 90% or more among the 75% or more stenosis group.

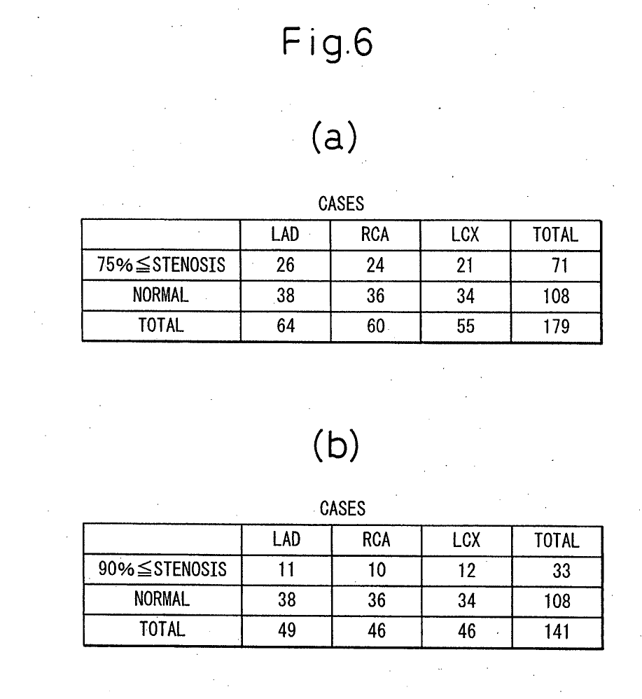

FIGS. 6(a) and 6(b) indicate the number of cases subjected to comparative studies. FIG. 6(a) is a table containing the 75% or more stenosis group (75%≦stenosis), while FIG. 6(b) is a table containing the 90% or more stenosis group (90%≦stenosis).

Comparative studies were conducted on 179 cases, of which 108 cases had normal coronary arteries while 71 cases had coronary artery stenosis of 75% or more. A breakdown of the cases with normal coronary arteries consists of 38 cases for LAD (group An), 36 cases for RCA (group Bn), and 34 cases for LCX (group Cn). A breakdown of the cases having coronary artery stenosis of 75% or more consists of 26 cases for LAD (group As), 24 cases for RCA (group Bs) and 21 cases for LCX (group Cs).

There were 33 cases that exhibited stenosis of 90% or more among the 71 cases of the 75% or more stenosis group. These consisted of 11 cases for LAD (group A's), 10 cases for RCA (group B's) and 12 cases for LCX (group C's).

Left ventricular wall motion was analyzed for these cases using the long-axis 2D strain rate method.

The region of interest (ROI, in this case the region where data is to be acquired) was first set by manually tracing the endocardium on the image. The width of the ROI was made to be narrow at roughly ⅓ the total layer on the side of the endocardium. The entire region of the ROI set at this time was confirmed to be able to be analyzed by the EchoPAC PC. If analysis was not possible, ROI setting was redone by returning to tracing of the endocardium followed by setting an ROI for which the entire region can be analyzed.

After designating the center of the anterior wall of the Ap2ch view as the LAD region, the center of the inferior wall as the RCA region, and the center of the posterior wall of the ApLax view as the LCX region, four factors consisting of the 100 ms value (1/s) (100 ms SR), 200 ms value (1/s) (200 ms SR), minimum SR value between 100 and 200 ms (1/s) and average SR value between 100 and 200 ms (1/s) of long-axis 2D strain rate (SR) were compared between groups An and As, groups Bn and Bs and groups Cn and Cs. Values were displayed as the mean ± variance, and “×10^{-x}” was displayed as “e−x”.

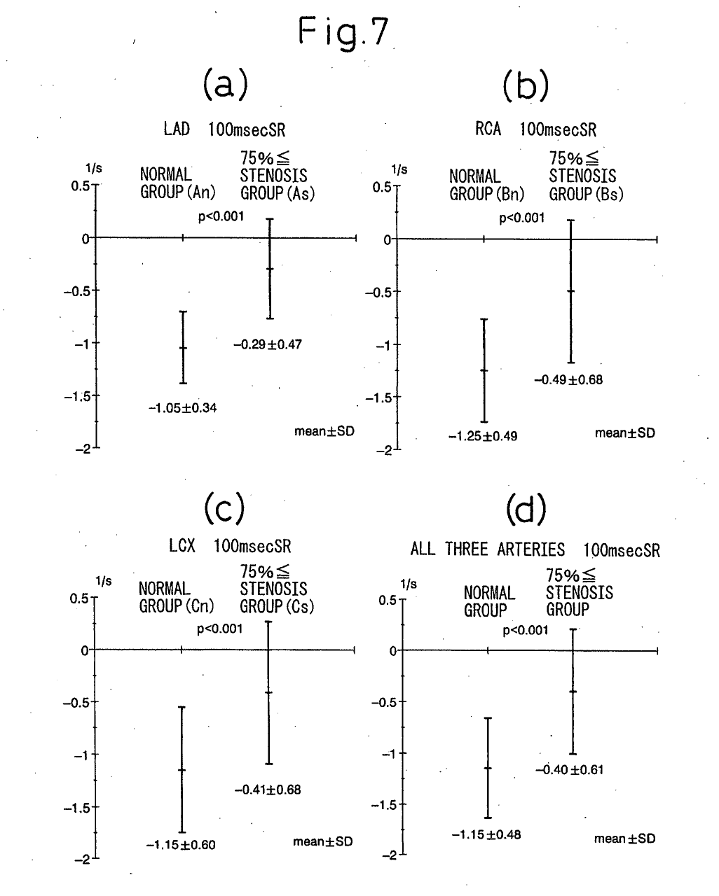

FIGS. 7 to 10 show the comparison results for the 75% or more stenosis group. The results for each of the four factors are collectively shown in FIGS. 7 to 10.

FIGS. 7(a) to 7(c) are drawings showing comparisons of 100 ms values (1/s) of long-axis 2D strain rate (SR).

As shown in FIG. 7(a), the 100 ms SR value for group An was −1.045±0.341, that for group As was −0.295±0.473, and the presence of a significant difference as determined according to the isovariance t-test (variance test) was P=4.724e−10.

As shown in FIG. 7(b), the 100 ms SR value for group Bn was −1.248±0.487, that for group Bs was −0.495±0.675, and the presence of a significant difference as determined according to the isovariance t-test (variance test) was P=5.144e−06.

As shown in FIG. 7(c), the 100 ms SR value for group Cn was −1.151±0.597, that for group Cs was −0.410±0.679, and the presence of a significant difference as determined according to the isovariance t-test (variance test) was P=9.024e−05.

FIG. 7(d) shows the results for all three arteries.

### The 100 ms SR value for the normal group was −1.145±0.484, that for the 75% or more stenosis group was −0.400±0.607, and the presence of a significant difference as determined according to the unisovariance t-test (Welch's t-test) was P=1.315e−14.

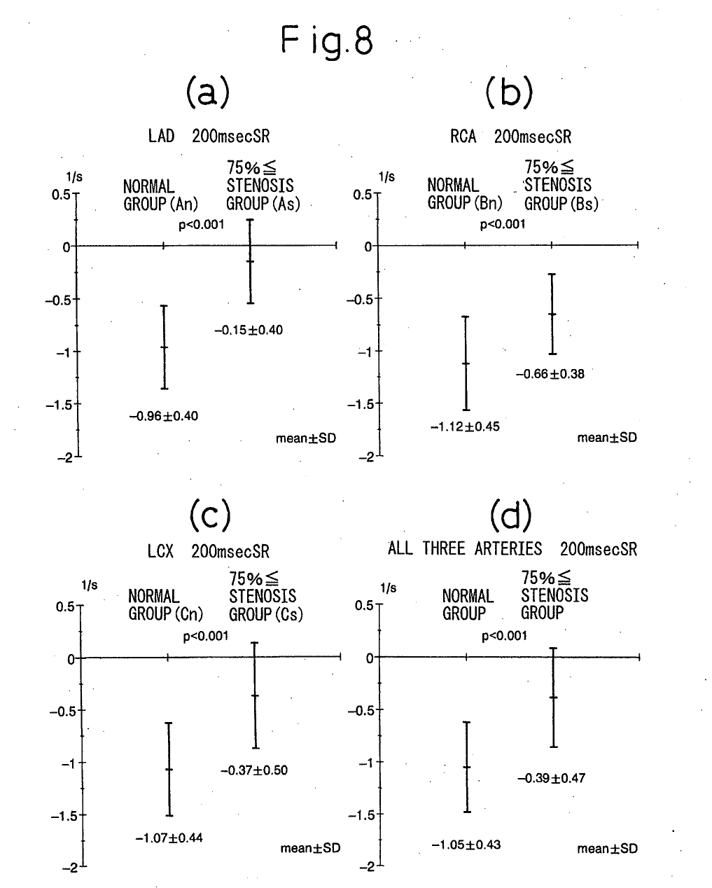

FIGS. 8(a) to 8(c) are drawings showing comparisons of 200 ms values (1/s) of long-axis 2D strain rate (SR).

As shown in FIG. 8(a), the 200 ms SR value for group An was −0.963±0.396, that for group As was −0.150±0.395, and the presence of a significant difference as determined according to the isovariance t-test (variance test) was P=2.928e−11.

As shown in FIG. 8(b), the 200 ms SR value for group Bn was −1.122±0.447, that for group Bs was −0.657±0.380, and the presence of a significant difference as determined according to the isovariance t-test (variance test) was P=9.435e−05.

As shown in FIG. 8(c), the 200 ms SR value for group Cn was −1.071±0.442, that for group Cs was −0.370±0.504, and the presence of a significant difference as determined according to the isovariance t-test (variance test) was P=1.509e−06.

FIG. 8(d) shows the results for all three arteries at a strain rate of 200 ms. The 200 ms SR value for the normal group was −1.050±0.429, that for the 75% or more stenosis group was −0.386±0.471, and the presence of a significant difference as determined according to the isovariance t-test was P=4.441e−16.

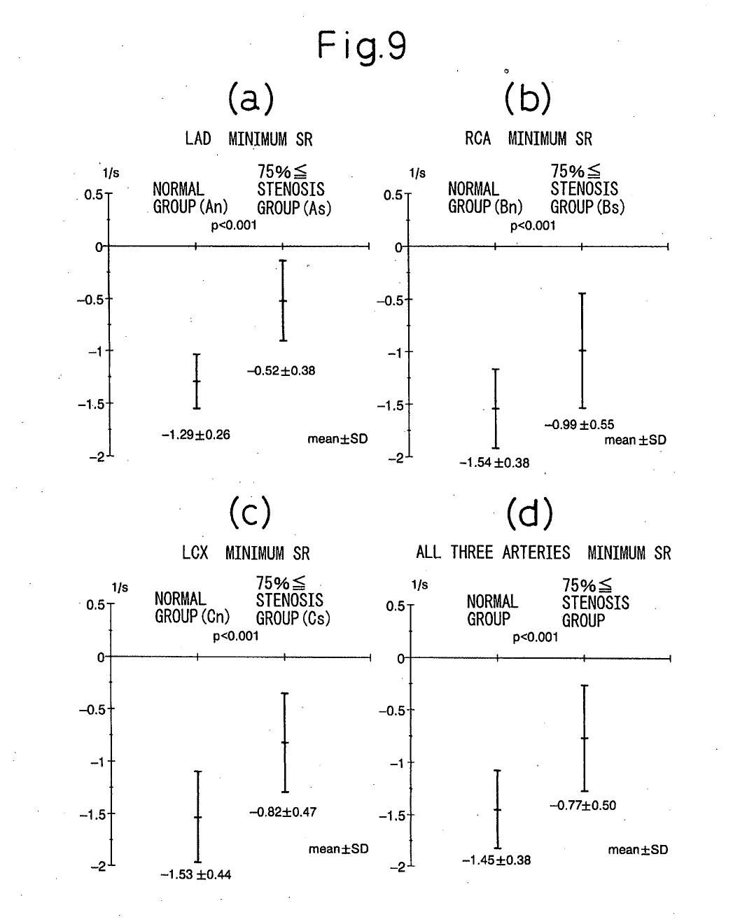

FIGS. 9(a) to 9(c) are drawings showing comparisons of minimum SR values (1/s) between 100 and 200 ms of long-axis 2D strain rate (SR).

As shown in FIG. 9(a), the minimum SR value for group An was −1.293±0.256, that for group As was −0.520±0.384, and the presence of a significant difference as determined according to the unisovariance t-test (Welch's t-test) was P=3.870e−11.

As shown in FIG. 9(b), the minimum SR value for group Bn was −1.541±0.375, that for group Bs was −0.986±0.547, and the presence of a significant difference as determined according to the unisovariance t-test (Welch's t-test) was P=0.0001.

As shown in FIG. 9(c), the minimum SR value for group Cn was −1.531±0.436, that for group Cs was −0.816±0.470, and the presence of a significant difference as determined according to the isovariance t-test was P=4.731e−07.

FIG. 9(d) shows the results for the minimum values of strain rate between 100 and 200 ms for all three arteries. The minimum SR value for the normal group was −1.451±0.375, that for the 75% or more stenosis group was −0.765±0.504, and the presence of a significant difference as determined according to the unisovariance t-test (Welch's t-test) was P=2.220e−16.

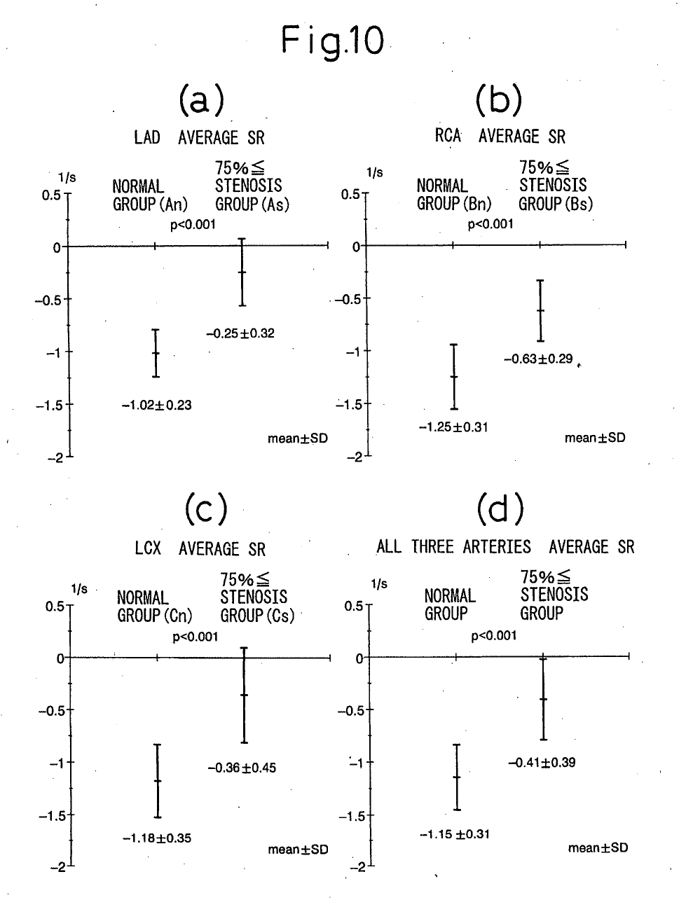

FIGS. 10(a) to 10(c) are drawings showing comparisons of average SR values (1/s) between 100 and 200 ms of long-axis 2D strain rate (SR).

As shown in FIG. 10(a), the average SR value for group An was −1.019±0.225, that for group As was −0.252±0.318, and the presence of a significant difference as determined according to the isovariance t-test was P=2.677e−16.

As shown in FIG. 10(b), the average SR value for group Bn was −1.250±0.305, that for group Bs was −0.626±0.291, and the presence of a significant difference as determined according to the isovariance t-test was P=8.694e−11.

As shown in FIG. 10(c), the average SR value for group Cn was −1.177±0.346, that for group Cs was −0.356±0.453, and the presence of a significant difference as determined according to the isovariance t-test was P=5.714e−10.

FIG. 10(d) shows the results for the average values of strain rate between 100 and 200 ms for all three arteries. The average SR value for the normal group was −1.146±0.307, that for the 75% or more stenosis group was −0.409±0.386, and the presence of a significant difference as determined according to the unisovariance t-test (Welch's t-test) was P=9.536e−17.

A receiver operating characteristic (ROC) curve was determined for the four factors of strain rate (SR) 100 ms value (1/s), 200 ms value (1/s), minimum SR value between 100 and 200 ms (1/s) and average SR value between 100 and 200 ms (1/s) based on the above data followed by determination of the borderline value (cutoff value) serving as the borderline between positive (stenosis) and negative (normal), sensitivity, which is an indicator for correctly judging positive results to be positive, and specificity, which is an indicator for correctly judging negative results to be negative.

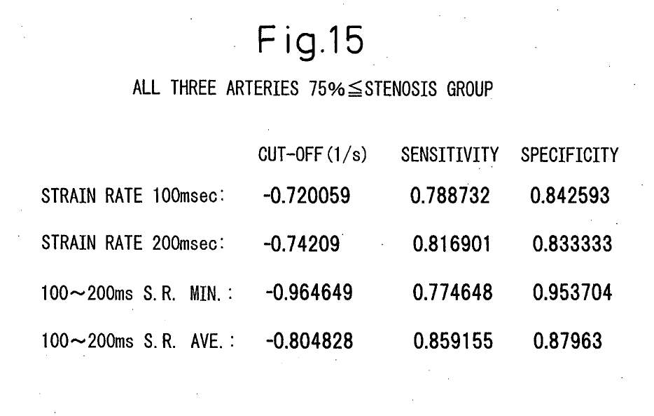

FIG. 15 shows the diagnosis rates for stenosis of 75% or more as determined from the optimum points on the ROC curve for all three arteries. The diagnosis rates for stenosis of 75% or more with respect to 100 ms SR value consisted of a cutoff value of −0.720, sensitivity of 78.9% and specificity of 84.3%. The diagnosis rates with respect to 200 ms SR value consisted of a cutoff value of −0.742, sensitivity of 81.7% and specificity of 83.3%, those with respect to minimum SR value consisted of a cutoff value of −0.965, sensitivity of 77.5% and specificity of 95.4%, and those with respect to average SR value consisted of a cutoff value of −0.805, sensitivity of 85.9% and specificity of 88.0%.

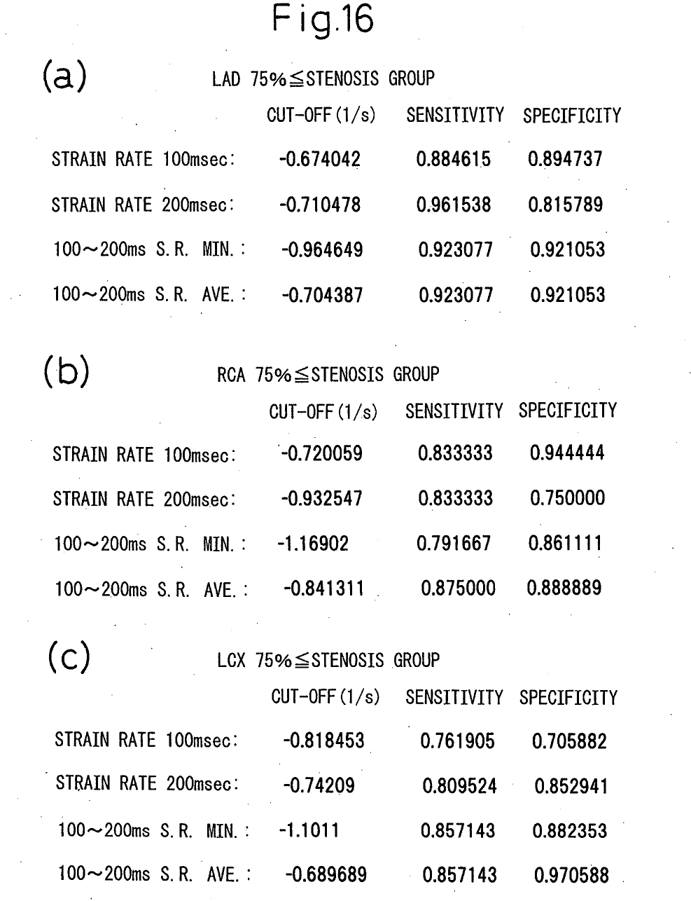

As shown in FIG. 16, satisfactory sensitivity and specificity were obtained for 100 ms SR, 200 ms SR, minimum SR and average SR values with respect to LCD, RCA and LCX as well.

Therefore, a discriminant function was generated using the four factors of strain rate consisting of the 100 ms value, 200 ms value, minimum SR value and average SR value. The resulting discriminant function using these four factors is shown below.

$$Discrimination score z = 4.91325 + 1.02145 \times ( 100 ms value ) + 1.2251 \times ( 200 ms value ) - 0.459876 \times ( minimum value ) + 4.82651 \times ( average value )$$

The detection probability was 0.86378 using Mahalanobis' generalized distance. Thus, if the detection probability is 86.39% and the discrimination score is z>0, there is judged to be significant stenosis of 75% or more, while if the discrimination score is z<0, there is judged to be stenosis of less than 75%.

When only the average value is used, the discrimination score becomes z=4.93841+6.35142×(average value) and the detection probability becomes 86.03%, thereby demonstrating that extremely effective results are obtained even when only using the average value.

However, it is necessary to use all of the data between 100 and 200 ms when calculating the average value. If only the three factors of strain rate 100 ms value, 200 ms value and minimum SR value are used while excluding the average value to simplify discrimination, the discriminant function becomes as follows:

$$Discrimination score z = 5.03099 + 2.16118 \times ( 100 ms value + 3.1742 \times ( 200 ms value ) + 0.978874 \times ( minimum value )$$

In practical terms, since 100 ms, 200 ms and minimum SR values can be acquired by displaying a graph of strain rate on a display device when using a discriminant function that excludes average value, the discrimination score can be calculated from this graph, thereby making this useful. The detection rate at this time was 85.28%.

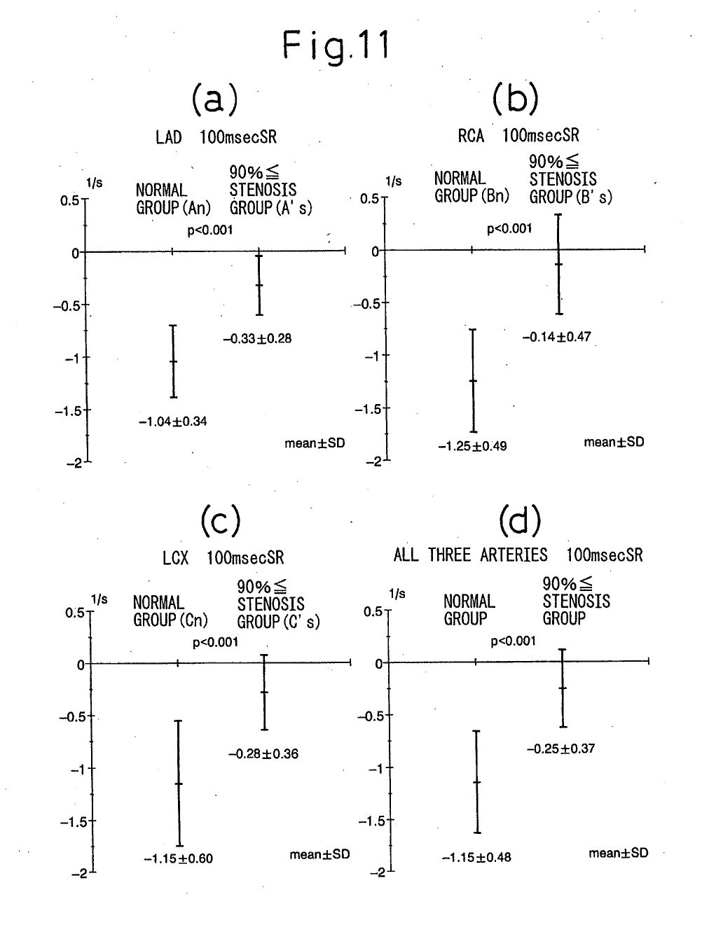

Next, a comparative study was conducted between the 33 cases of the 71 cases in the stenosis group having stenosis of 90% or more and the 108 normal cases. As was previously described, these consisted of 11 cases for LAD (group A's), 10 cases for RCA (group B's) and 12 cases for LCX (group C's). The results of the comparison are shown in FIGS. 11 to 14.

FIGS. 11 to 14 show a comparison of cases having 90% or more stenosis and normal cases for the four factors of long-axis 2D strain rate (SR) 100 ms values (1/s) (100 ms SR), 200 ms values (1/s) (200 ms SR), minimum SR value between 100 and 200 ms (1/s) and average SR value between 100 and 200 ms (1/s) in the same manner as in the case of stenosis of 75% or more. Values for LDA, RCA and LCX in the normal group were designated as An, Bn and Cn, while values for the 90% or more stenosis group were designated as A's, B's and C's. In addition, values were displayed as the mean ± variance, and “×10^{-x}” was displayed as “e−x”. Furthermore, the data for An, Bn and Cn of the normal group is omitted since it was previously explained.

FIGS. 11(a) to 11(c) are drawings showing comparisons of 100 ms values (1/s) of long-axis 2D strain rate (SR) between the 90% or more stenosis group and the normal group.

As shown in FIG. 11(a), the 100 ms SR value for LAD in the A's group indicating stenosis was −0.327±0.280, and the presence of a significant difference with the normal group as determined according to the isovariance t-test was P=7.281e−08. As shown in FIG. 11(b), the 100 ms SR value for RCA in the B's group indicating stenosis was −0.142±0.474, and the presence of a significant difference with the normal group as determined according to the isovariance t-test was P=8.396e−08. As shown in FIG. 11(c), the 100 ms SR value for LCX in the C's group indicating stenosis was 0.283±0.359, and the presence of a significant difference with the normal group as determined according to the isovariance t-test was P=2.422e−05. As shown in FIG. 11(d), the results of a comparison of 100 ms SR values for all three arteries yielded a value of 0.254±0.372, and the presence of a significant difference as determined according to the isovariance t-test was P=1.110e−16.

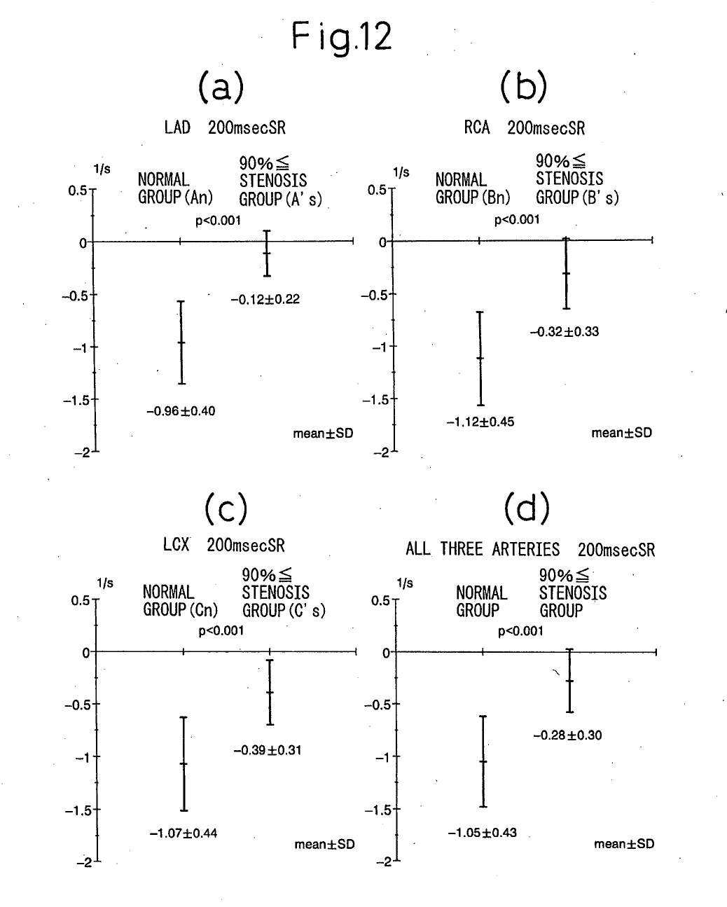

FIGS. 12(a) to 12(c) are drawings showing comparisons of 200 ms values (1/s) of long-axis 2D strain rate (SR) between the 90% or more stenosis group and the normal group.

As shown in FIG. 12(a), the 200 ms SR value for LAD in the A's group indicating stenosis was −0.115±0.216, and the presence of a significant difference with the normal group as determined according to the unisovariance t-test was P=1.868e−10. As shown in FIG. 12(b), the 200 ms SR value for RCA in the B's group indicating stenosis was −0.316±0.332, and the presence of a significant difference with the normal group as determined according to the isovariance t-test was P=3.562e−06. As shown in FIG. 12(c), the 200 ms SR value for LCX in the C's group indicating stenosis was −0.391±0.308, and the presence of a significant difference with the normal group as determined according to the isovariance t-test was P=1.292e−05. As shown in FIG. 12(d), the results of a comparison of 200 ms SR values for all three arteries yielded a value of 0.276±0.304, and the presence of a significant difference as determined according to the unisovariance t-test (Welch's t-test) was P=1.290e−16.

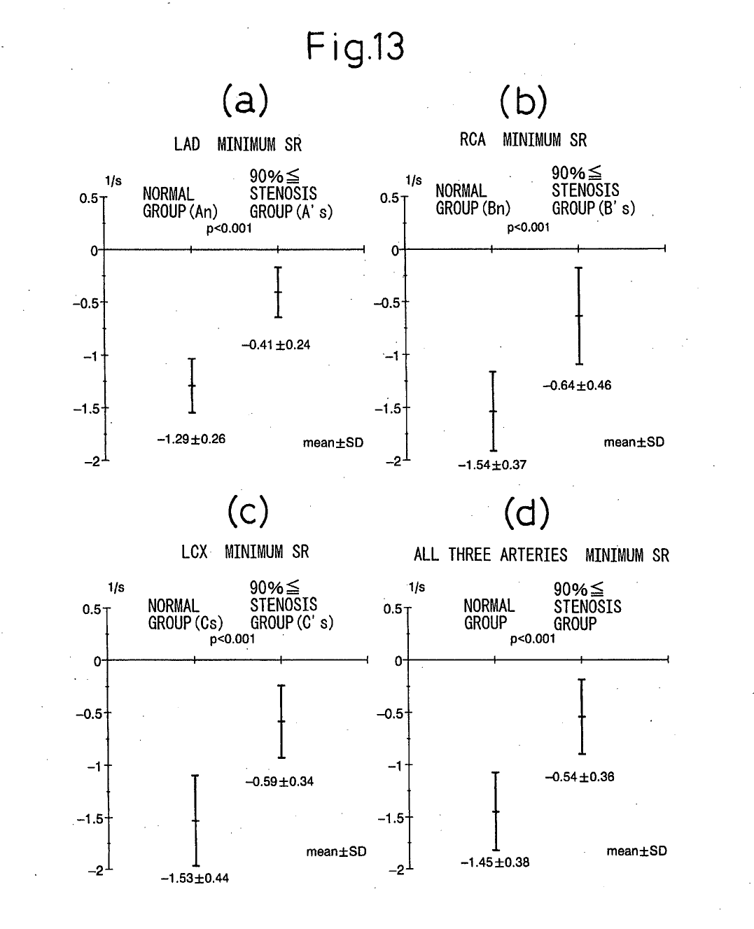

FIGS. 13(a) to 13(c) are drawings showing comparisons of minimum SR values (1/s) between 100 and 200 ms of long-axis 2D strain rate (SR) between the 90% or more stenosis group and the normal group.

As shown in FIG. 13(a), the minimum SR value for LAD in the A's group indicating stenosis was −0.407±0.239, and the presence of a significant difference with the normal group as determined according to the isovariance t-test was P=1.501e−13. As shown in FIG. 13(b), the minimum SR value for RCA in the B's group indicating stenosis was −0.640±0.459, and the presence of a significant difference with the normal group as determined according to the isovariance t-test was P=8.396e−08. As shown in FIG. 13(c), the minimum SR value for LCX in the C's group indicating stenosis was −0.587±0.344, and the presence of a significant difference with the normal group as determined according to the isovariance t-test was P=2.374e−08. As shown in FIG. 13(d), the results of a comparison of minimum SR values for all three arteries yielded a value of 0.543±0.357, and the presence of a significant difference as determined according to the isovariance t-test was P=4.441e−16.

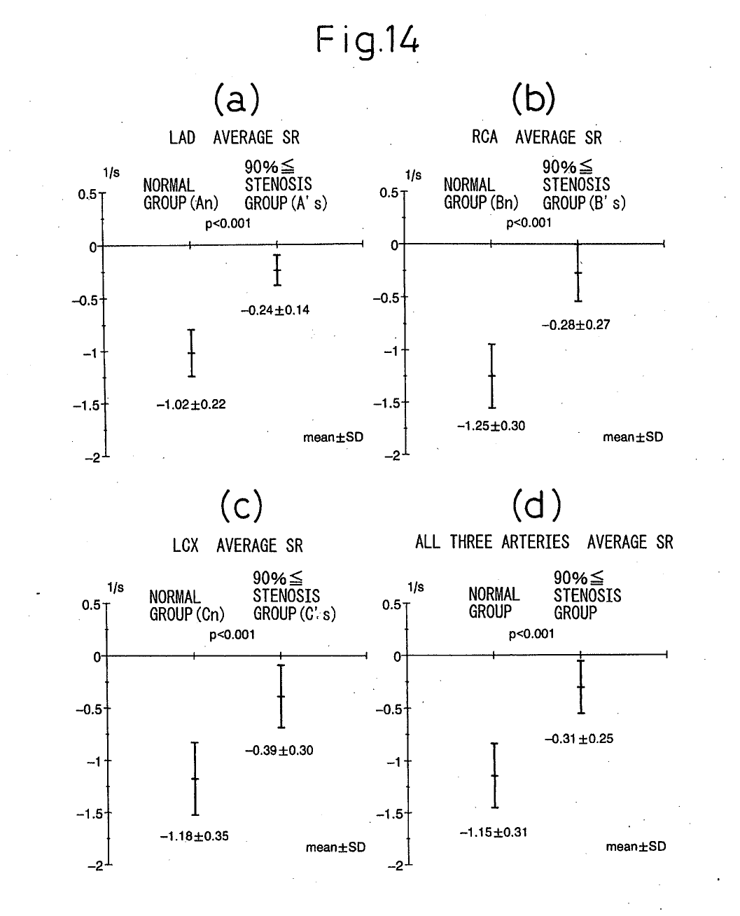

FIGS. 14(a) to 14(c) are drawings showing comparisons of average SR values (1/s) between 100 and 200 ms of long-axis 2D strain rate (SR) between the 90% or more stenosis group and the normal group.

As shown in FIG. 14(a), the average SR value for LAD in the A's group indicating stenosis was −0.242±0.143, and the presence of a significant difference with the normal group as determined according to the isovariance t-test was P=2.376e−14. As shown in FIG. 14(b), the average SR value for RCA in the B's group indicating stenosis was −0.275±0.271, and the presence of a significant difference with the normal group as determined according to the isovariance t-test was P=9.516e−12. As shown in FIG. 14(c), the average SR value for LCX in the C's group indicating stenosis was −0.388±0.298, and the presence of a significant difference with the normal group as determined according to the isovariance t-test was P=1.080e−08. As shown in FIG. 14(d), the results of a comparison of average SR values for all three arteries yielded a value of 0.305±0.249, and the presence of a significant difference P as determined according to the isovariance t-test was P=0.000.

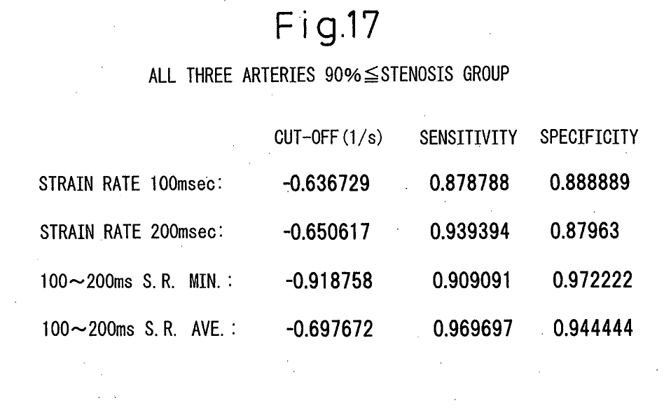

FIG. 17 shows the results of determining the borderline values (cutoff values), sensitivity and specificity from the optimum points of the ROC curve based on data for all three arteries for stenosis of 90% or more.

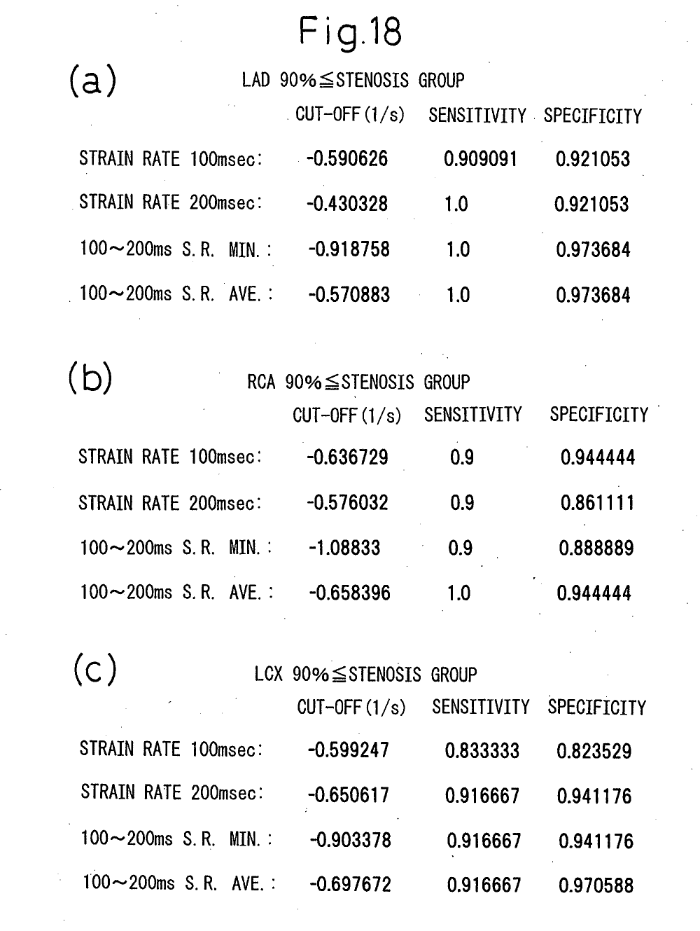

In the case of 100 ms SR, the cutoff value was −0.637, sensitivity was 87.9% and specificity was 88.9%. In the case of 200 ms SR, the cutoff value was −0.637, sensitivity was 99.9% and specificity was 88.0%. In the case of minimum SR, the cutoff value was −0.919, sensitivity was 90.9% and specificity was 97.2%. In the case of average SR, the cutoff value was −0.698, sensitivity was 97.0% and specificity was 94.9%. In addition, as shown in FIG. 18, sensitivity and specificity for each of the three arteries was even better than that for stenosis of 75% or more using all four factors.

Next, a discriminant function was generated for stenosis of 90% or more using the four factors of strain rate consisting of the 100 ms value, 200 ms value, minimum SR value and average SR value. The resulting discriminant function using these four factors is shown below.

$$Discrimination score z = 8.20393 + 2.18254 \times ( 100 ms value ) + 3.1092 \times ( 200 ms value ) + 2.36328 \times ( minimum value ) + 3.11278 \times ( average value )$$

The detection probability was 0.934414 using Mahalanobis' generalized distance. Thus, if the detection probability is 93.44% and the discrimination score is z>0, there is judged to be significant stenosis of 90% or more, while if the discrimination score is z<0, there is judged to be stenosis of less than 90%.

In addition, if only the three factors of strain rate 100 ms value, 200 ms value and minimum SR value are used while excluding the average value, the discriminant function becomes as follows:

$$Discrimination score z = 8.18357 + 2.96442 \times ( 100 ms value + 4.33182 \times ( 200 ms value ) + 3.24544 \times ( minimum value ) ,$$

and the detection factor was 93.25%.

As has been described above, favorable significant differences, sensitivity and specificity were observed for all three coronary artery regions for the four factors of 100 ms SR value, 200 ms SR value, minimum SR value between 100 and 200 ms and average SR value between 100 and 200 ms. In particular, the average SR value was the most useful factor. As a result of adding a plurality of factors between 100 and 200 ms (here, 100 ms SR value, 200 ms SR value and minimum SR value between 100 and 200 ms) to the average SR value between 100 and 200 ms, the detection rate for stenosis of 75% or more was 86.39% while that for stenosis of 90% or more was 93.44%.

As a result of analyzing resting left ventricular wall motion by suitably modifying the setting of ROI using the long-axis 2D strain rate method in echocardiography, angina pectoris was able to be diagnosed at a probability roughly equal to that of stress echocardiography. In addition, although assessment of the severity of coronary artery stenosis is difficult to be diagnosed by conventional stress echocardiography, the use of a plurality of factors as used in the present invention to calculate a discriminant function made it possible to make such a diagnosis.

What is claimed is:

1. An apparatus for diagnosing ischemic heart disease comprising:
a unit for displaying resting apical long-axis tomographic images obtained by an echocardiography imaging unit;
a region of interest setting unit for setting the region of interest at a thin layer on the inside of the left ventricular wall in the long-axis echocardiographical images;
a strain rate calculation unit for calculating the strain rate of the region of interest; and
a discrimination value calculation unit for calculating the value of a predetermined discriminant function based on a plurality of strain rates of an intermediate portion of the systolic phase; wherein,
ischemic heart disease is diagnosed according to the calculated discrimination value.

2. The apparatus according to claim 1, wherein the width of the thin layer on the inside of the left ventricular wall is preferably ⅓ or less the entire left ventricular wall layer.

3. The apparatus according to claim 1, wherein the intermediate portion of the systolic phase is within the range of 100 to 200 ms.

4. The apparatus according to claim 1, further comprising a unit that calculates an average value from the plurality of strain rates at an intermediate portion of the systolic phase, and
ischemic heart disease is diagnosed based on that average value.

5. The apparatus according to claim 1, wherein the plurality of strain rates of an intermediate portion of the systolic phase consist of the strain rate at the start of the intermediate portion of the systolic phase, the strain rate at the end of the intermediate portion, and the minimum value of the strain rate of the intermediate portion.

6. The apparatus according to claim 4, wherein ischemic heart disease is diagnosed based on the average value as well as the strain rate at the start of the intermediate portion of the systolic phase, the strain rate at the end of the intermediate portion, and the minimum value of the strain rate of the intermediate portion.

7. A method for diagnosing ischemic heart disease comprising:
setting a region of interest at a thin layer on the inside of the left ventricular wall for echocardiographical apical long-axis tomographic images obtained while at rest;
calculating the strain rate of the set region of interest;
calculating a predetermined discriminant function on the basis of a plurality of strain rates of an intermediate portion of the systolic phase; and
diagnosing ischemic heart disease according to that discrimination value.

8. The method according to claim 7, wherein the width of the thin layer on the inside of the left ventricular wall is preferably ⅓ or less the entire left ventricular wall layer.

9. The method according to claim 7, wherein the intermediate portion of the systolic phase is within the range of 100 to 200 ms.

10. The method according to claim 7, further comprising calculation of an average value from the plurality of strain rates at an intermediate portion of the systolic phase, and
diagnosing ischemic heart disease based on that average value.

11. The method according to claim 7, wherein the plurality of strain rates of an intermediate portion of the systolic phase consist of the strain rate at the start of the intermediate portion of the systolic phase, the strain rate at the end of the intermediate portion, and the minimum value of the strain rate of the intermediate portion.

12. The method according to claim 10, wherein ischemic heart disease is diagnosed based on the average value as well as the strain rate at the start of the intermediate portion of the systolic phase, the strain rate at the end of the intermediate portion, and the minimum value of the strain rate of the intermediate portion.

13. A storage medium having stored thereon a computer program executable to perform the steps of:
setting a region of interest at a thin layer on the inside of the left ventricular wall for ecocardiographical apical long-axis tomographic images obtained while at rest;
calculating the strain rate of the set region of interest; and
calculating the value of a predetermined discriminant function based on a plurality of strain rates of an intermediate portion of the systolic phase.

14. The storage medium according to claim 13, wherein the width of the thin layer on the inside of the left ventricular wall is preferably ⅓ or less the entire left ventricular wall layer.

15. The storage medium according to claim 13, wherein the intermediate portion of the systolic phase is within the range of 100 to 200 ms.

16. The storage medium according to claim 13, further comprising calculating an average value from the plurality of strain rates at an intermediate portion of the systolic phase, and
diagnosing ischemic heart disease based on that average value.

17. The storage medium according to claim 13, wherein the plurality of strain rates of an intermediate portion of the systolic phase consist of the strain rate at the start of the intermediate portion of the systolic phase, the strain rate at the end of the intermediate portion, and the minimum value of the strain rate of the intermediate portion.

18. The storage medium according to claim 16, wherein ischemic heart disease is diagnosed based on the average value as well as the strain rate at the start of the intermediate portion of the systolic phase, the strain rate at the end of the intermediate portion, and the minimum value of the strain rate of the intermediate portion.
